(19) United States
(12) Patent Application Publication
(10) Pub. No.: US 2017/0101196 A1
(43) Pub. Date: Apr. 13, 2017

(54) METHOD FOR PROCESSING OXYGEN FROM CARBON DIOXIDE ABOARD HUMAN SPACECRAFTS AND METHANE FROM CARBON DIOXIDE AND OXIDE MINERALS FOUND ON MARS AND OTHER CELESTIAL BODIES

(71) Applicant: Angelo Karavolos, Spring, TX (US)

(72) Inventor: Angelo Karavolos, Spring, TX (US)

(21) Appl. No.: 15/207,474

(22) Filed: Jul. 11, 2016

Related U.S. Application Data
(60) Provisional application No. 62/191,167, filed on Jul. 10, 2015.

(51) Int. Cl. B64G 1/48; C07C 1/12; C01B 25/37; C01B 3/06; C01B 13/02

(52) U.S. Cl. CPC B64G 1/48; C01B 3/06; C01B 13/0203; C01B 25/37; C07C 1/12

(57) ABSTRACT

Method suitable for production of oxygen and methane from carbon dioxide and oxide minerals transported aboard human-occupied spacecraft or found on Mars or other celestial bodies with use of dodecatungstophosphoric acid, which is regenerated with the use of acids such as phosphoric acid and hydrochloric acid, which is then mixed with sodium tungstenate. Embodiment using a hydrogenated large complex polymer formed of polycarbonate and PO₄W₁₂O₃₆.

### BACKGROUND OF THE INVENTION

1. Field of the Invention

The present invention relates generally to the field of recycling and regenerating materials consumed during extended human space flight, more specifically to the use of a metal oxide-containing compound for such purpose, and especially to the use of such chemistry when supplied with crew-generated waste products or with materials extracted from an atmosphere or surface of a planet such as Mars or of another celestial body.

2. General Background and State of the Art

Space flight undertaken over long distances and for prolonged periods tends to require consumable materials in quantities not practicably deliverable with contemporary propulsion systems. Limitations on mass, energy and volume that can be delivered to a time and place of need dictate that materials be conserved, recycled, or extracted from local sources and the like.

### BRIEF DESCRIPTION OF THE DRAWINGS

For a further understanding of the objects and advantages of the present invention, reference should be had to the following detailed description, taken in conjunction with the accompanying drawing, in which like parts are given like reference numbers and wherein:

FIG. 1 is a schematic rendering of A FIRST EXEMPLARY EMBODIMENT of the PROCESSING METHOD in accordance with the present invention

### DETAILED DESCRIPTION OF THE PREFERRED EMBODIMENTS

1 kg dodecahedral tungsten phosphate hydrate, “DTPA” chemical formula H₃PW₁₂O₃₆.59 H₂O, is provided by performing the following chemical reaction, the H3PO4 W12O40 product of which precipitates:

H₃PO₄+W₁₂O₄₀.59H₂O→H₃PO₄ W₁₂O₄₀+59 H₂O

Physical steps undertaken to accomplish this reaction include the addition of phosphoric acid, hydrochloric acid, and sodium hydroxide to a metallic residue, a step of mixing, and a step of injecting the product between membranes within a pressure vessel (described below).

The starting form of this material is a dodecahedron crystal delivered as a finely divided particulate capable of being suspended in air or in a solgel. The particle size is approximately 10-20 microns.

A TEOS solgel is mixed with the finely divided DTPA. The mixture is decanted, centrifuged and dried.

Containment is provided by a pressure vessel formed from, e.g., HDPE, carbon fiber or steel.

Support structure within the pressure vessel includes a filter wafer material made of DTPA mixed with sol gel, contained between thin porous membranes.

The pressure vessel is depressurized to less than ambient pressure. On Mars, low pressure conditions already exist.

Temperature is ambient or slightly below. On Mars, low temperature conditions already exist.

In anticipation that the reaction will generate heat, the pressure vessel and its contents are kept cold, so that when heat is generated within the pressure vessel, the heat will migrate from where it is generated to a cooler place within the pressure vessel. A heat sink inside is included within the pressure vessel. The heat sink may be formed of an otherwise suitable material having a high heat capacity. The estimated mass of the heat sink is about 1 kg on the scale of this exemplary embodiment.

Electrical charge is likely to build up, possibly sufficient to provide a current of 0.001 A to 1.0 A during periods of peak reaction.

The components needed for this effort are a large pressure container made of high density polyethylene, stainless steel, or carbon fiber composite; three pressure regulators with outtake and intake ports for each; a set of filters which can be inserted and removed from the pressure vessel; and five collection chambers for oxygen gas, methane cas, phosphoric acid, HCO₃, and water.

The pressure vessel will have at least five ports:

#1 negative electrical lead

#2 positive electrical lead

#3 inlet valve for fluids: acidified water in

#4 inlet valve for fluids: acidified water out

#5, 6 heat extraction loop, utilizing a recirculating fluid

#7 injector for DTPA-sol-gel during recharge step if needed

The process is begun by adding CO₂ to the pressure vessel at a time when the pH is between 6.5 and 8.0. The chemical reaction is carried out at constant temperature and pressure and is described as follows:

H₃PO₄.W₁₂O₃₆.59 H₂O+12 CO₂+12 H₂O

. . . →12 HCO₃+H₃PO₄W₁₂O₃₆.59 H₂O

. . . →PO₄⁻²_{aq}+W₁₂O₃₆⁺⁴.59 H₂O_{diss}+12 CO₃⁻²_{aq}+15/2 H₂^{↑}

The subscript “aq” means “aqueous,” “diss” means “dissolved,” and the arrow pointing upward signifies a gas evolving from the reaction.

Additionally, the following reaction occurs:

3/2 H₂+PO₄-2+W₁₂O₃₆

→W₁₂O₃₆*59H₂O→ΔH→W₁₂O₃₆+59H₂O??

The physical step for accomplishing the above reaction is the opening of an intake valve to admit CO₂ to the pressure vessel, whereupon the reaction proceeds spontaneously.

Next, water is removed, triggering the next stage of the process in accordance with the following equations:

PO₄²⁻+4 NaOH+W₁₂O₃₆+59H₂O+12CO₃

. . . →_{pressure drop} NAPO_{4 s}+NA₂CO_{3 s}+W₁₂O₃₆+59 H₂O^{↑}+18 O_{2 g}^{↑}

Next, O₂ is outgassed, triggering the next stage of the process in accordance with the following chemical reactions:

NA₂O₄+NA₂CO₃→4 H₂O

. . . →_{constant temperature and pressure; precipitation} H₃PO₄+HCO₃+4 NaOH+2 O₂

12 WO₃→_{constant T and dropping P} 13 O₂+12 Wₛ

In the wafers, in the sol gel, two phases form, including crystallized WO₄. Crystals of WO₄ collect at the bottom; acidified water is formed in the supernatant aqueous phase. The sorting out of materials is gravity-driven and also can be electrostatically driven (gravity on Mars; electrostatic in a microgravity environment).

A recharging process in accordance with the present invention proceeds in accordance with the following reactions:

2 NaOH→apply heat Na₂O+H₂O

12 H₂O+12 CO_{2 g}→12 HCO₃ stored

12 HCO_{3 aq}+6 Na₂PO_{4 aq stored}→Na₂CO₃+H₃PO₄

12W+12 Na₂O+36 OH→_{heat with NA2O}→12 Na₂WO₄.2 H₂O+18 H₂

12 Na₂WO₄.2 H₂O+12 HCl+H₃PO₄→H₃PO₄W₁₂O₃₆.2 H₂O+12 NaCl+6 H₂

H₃PO₄W₁₂O₃₆+59 H₂O→H₃PO₄W₁₂O₃₆.59H₂O

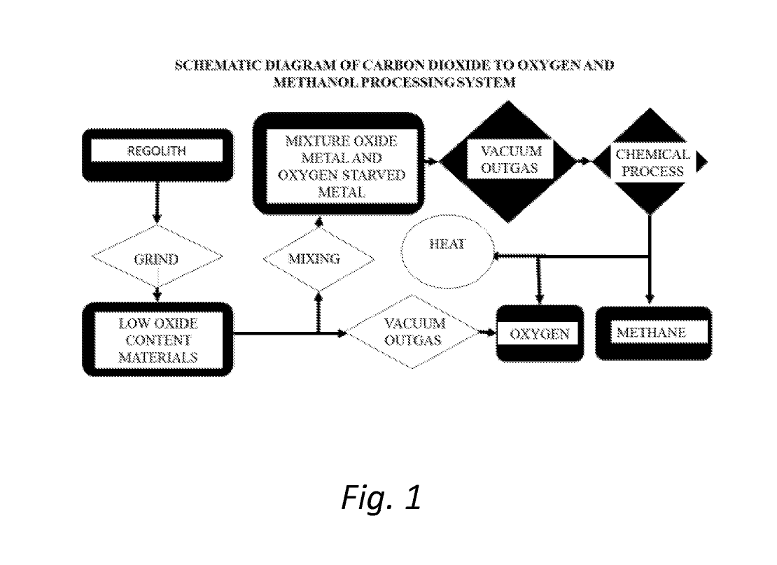

FIG. 1 is a schematic rendering of a first exemplary embodiment of the processing method in accordance with the present invention, showing that regolith gathered from a surface of a celestial body is ground and the resulting low oxide-content material is mixed with acid such as phosphoric acid, hydrochloric acid, and sodium hydroxide to produce a mixture of oxide metal and oxygen starved metal.

In a second exemplary embodiment, polycarbonate can be blended with PO₄W₁₂O₃₆ to form a large complex polymer as follows:

1. Blend polycarbonate with PO₄W₁₂O₃₆ to form a large complex polymer.

2. Hydrogenate this polymer.

3. Introduce this polymer into CO₂ to form bicarbonates at the end of each tungsten site of the dodecahedron.

Blend the resulting mixture with poly-carbonate, a polymer known to release Oxygen when exposed to UV radiation. By blending the mixture with W₁₂O₃₆, we give it 36 separate sites with which to out-gas oxygen. This oxygen comes from the CO₂ it combined with, not from water.

The following materials were prepared in order to conduct the experimental procedure.

These materials include dodeca tungsto phosphoric acid, tetra ethyl ortho silicate, and polycarbonate polymer. Other materials needed are distilled water, and NaOH buffer solution.

### Material Preparation

### Dodeca Tungsto Phosphoric Acid

For the deposition of thin films of phosphoric tungstic acid [H₃(PW₁₂O₄₀)], 2% solution of phosphor tungstic acid in distilled water was taken in 200 cm³ glass beaker. The speed of substrate rotation was kept 200 rpm. After half hour, there was white colored and uniform deposition of [H₃(PW₁₂O₄₀)] on polyaniline film 2 mm thick. As deposited thin films were dried in constant temperature oven at 80° C. After cooling at room temperature, these films were dipped in 0.1% aqueous solution of polyacrylamide (PAM) in order to get the adhesive thin films.

### Dodeca Tungsto Phosphoric Acid-Polycarbonate

Prepare a 5% solution of phosphoric tungstic acid and double distilled water, then mixed this solution to 15% tetra ethyl ortho silicate, in a 200 cm3 beaker. The speed of the mixing was set at 2000 rpm for 30 seconds, in 10 second intervals, and 5 second intermission periods to avoid over heating the specimen.

The mixture above was allowed to set for 1 hour at room temperature, and kept in a desiccator to eliminate water adsorption. Upon completion of this task the mixture, will be added to a 5% solution of polycarbonate. This solution is also mixed at 2000 rpm for 30 seconds, allowing for 10 second intermissions in order to dissipate heat released during the mixing process.

Before the polycarbonate is mixed with the tetra ethyl ortho silicate, it is mixed with the dodeca tungsto phosphoric acid and heated so that it will polymerize in a step growth process. The operating temperature for the polymerization should be held constant at 155° C., well below the melting temperature of dodeca tungsto phosphoric acid.

### Tetraethyl Orthosilicate (TEOS):

The matrix material tetraethyl orthosilicate is prepared.

Weigh 2.00 g NH4F and add it to 100 mL of distilled water.

Mix 5.0 g TEOS and 10.0 g ethanol in a 250 ml beaker.

Mix 10.0 g water and 15.0 g ethanol in another beaker.

Pour the catalyst solution into the solution and stir at 200 rpm for 30 seconds.

Place the material in a desired mold (10 cm by 10 cm by 2 mm), and store in a desiccator at room temperature and 1 atmosphere of pressure.

### Experimental Method

A first experiment tests the hypothesis that dodeca tungsto phosphoric acid can be decomposed, through interaction with CO₂ and reduction of pressure, to WO₃. A second experiment tests the hypothesis that WO₃ can be recycled back to dodeca tungsto phosphoric acid. A third experiment can trace the pathway of O₂, from the introduction of CO₂ and the decomposition of dodeca tungsto phosphoric acid, to the formation of dodeca tungsto phosphoric acid from WO₃.

Three experiments were completed in order to validate (or invalidate) the hypothesis of the experiment. The first experiment involved establishing controls for making sure all interpretations of the data involved the same set of assumptions and baseline. Another set of experiments were completed which involved releasing measured known amounts of CO2 gas to the filter material, and weighing the resulting O2 produced. Oxygen gas was also measured before and after CO2 was released to the dielectric filter. Finally, the weight of CO2, becoming HCO3, was measured, so that a complete mass balance evaluation of the system can be completed.

An assumption was made that reactions taking place within the chamber are occurring in a homogeneous mixture. To facilitate homogeneous mixing, the chamber shall rest upon a magnetic stirring appliance. The second assumption was that all of the CO2 shall be consumed in the process, and all of the O₂ contained in the super oxide moves into solution upon decompression, Based upon work by Hsu et. al, (1996), the splitting of O₂ from HCO₃ cannot be sustained thermodynamically. However, W(HCO₃)₁₂—O₂ release can occur. The strategy of these experiments shall be to determine the relationship between the change in pH (independent variable) and the molar concentration of gas released per unit volume.

A test chamber constructed to test the material comprised three containers. Carbon dioxide gas flowed from the container to the capacitor chamber. Carbon dioxide flowed into the dielectric material. The pH of the mixture was reduced by the addition of CO₂, and the chamber depressurized to 0.5 atmosphere. O₂ gas then outgassed into the chamber.

Two additional control samples were prepared, one that had no TEOS with the O₂ rich metal material and another control that had no O₂ rich metal material and TEOS material. Background O₂ levels were measured with a dissolved O₂ meter, and CO2 was measured with a CO₂ gas meter.

It is desirable to know what the source of O₂ was for the regenerated DTPA. Using a tracer molecule such as O₁₈ the concentration of this tracer can be measured before and after the processes of degeneration of DTPA, formation of WO₃, and back to DTPA as follows:

1. Place a small amount of WO₃ in a 200 ml beaker. Mix with 100 grams of distilled water.

2. Add to this mixture, 50 mg of Na₃WO₃.

3. Add H3PO4 to the mixture of (2). Sodium phosphor-tungstate-Na₃PO₄W₁₂O₃₆ shall precipitate.

4. Decant this precipitate, and titrate the solution with HCl. A precipitate will form, H₃PO₄W₁₂O₃₆. Accelerate this precipitation by applying a specified pressure to the system.

5. Place a small amount of tungsten super oxide, H₃PO₄W₁₂O₃₆, (DTPA) in a 200 ml beaker.

6. Add to this powder, 100 gm of distilled water.

7. Add a known amount of CO₂, at a ratio of 1760 grams of gas to 2880 grams of DTPA.

8. Add sufficient amount of CO₂ to the solution to increase the pH from 2.0 to 8.0. Monitor using a pH monitor.

9. Using a thermocouple probe inserted within the dielectric, and a voltmeter connected to the conductive layers of the capacitor chamber, measure the temperature, heat, and voltage of the capacitor chamber. These values can empirically determine how much DTPA can be converted to WO₃.

10. Using X-ray diffraction, nuclear magnetic resonance, and cyclotron analysis, determine the amount of WO₃ in a sample of the dielectric material.

These experiments, in conjunction with chemical modeling support the conclusion that DTPA can be broken down to WO₃, O₂ released, CO₂ adsorbed, and DTPA reformed using specified amounts of Na₃WO₃, H₃PO₄, and HCl. Examples are presented below:

1. Start with Tungsto phosphoric acid, O₁₈, H₃, H₂O distilled, Sodium Tungstate, Sigma Aldrich.

2. Provide Mass Spectrometer, XRD, NMR, Voltmeter, pH meter, thermocouple. Deconstruct Dodeca Tungsto Phosphoric Acid (DTPA).

2a. Place 50 mg DTPA in 200 ml Erlenmeyer Flask, along with 50 mg Sodium Tungstate, in 200 ml distilled water.

2b. Combine this mixture with 100 mg Tetra Ethyl Ortho Silicate in a 500 ml Erlenmeyer Flask.

2c. Mix this with an additional 200 ml distilled water using magnetic stir, 200 rpm, for 15 minutes. For species tracing purposes, use either O₁₈ or H.

3. Evaluate samples at stages (2a, 2b, 2c) using XRD, Cyclotron, NMR, Mass Spectroscopy Analysis. Prepare controls of each (DTPA, Sodium Tungstate, Tungsten Oxide) for baseline comparison.

These two experiments encompass the validation or invalidation of the action of H₃PO₄W₁₂O₃₆ producing O₂, and adsorbing CO₂ producing W(HCO₃)₁₂. Thirdly, the last experiment validated the formation of H₃PO₄W₁₂O₃₆ from WO₃.

As can be seen from the drawing figures and from the description, each embodiment of the device in accordance with the present invention solves a problem by addressing the need for removal of carbon dioxide and supplying consumables—in some embodiments, without introducing additional raw materials, but instead drawing from the broken down materials from the reaction.

While the specification describes particular embodiments of the present invention, those of ordinary skill can devise variations of the present invention without departing from the inventive concept.

Although specific embodiments have been illustrated and described herein, those of ordinary skill in the art will appreciate that any arrangement calculated to achieve same purposes can be substituted for the specific embodiments shown. This disclosure is intended to cover any and all adaptations or variations of various embodiments of the invention. It is to be understood that the above description has been made in an illustrative fashion, and not a restrictive one. Combinations of the above embodiments, and other embodiments not specifically described herein will be apparent to those of skill in the art upon reviewing the above description. The scope of various embodiments of the invention includes any other applications in which the above structures and methods are used. Therefore, the scope of various embodiments of the invention should be determined with reference to the appended claims, along with the full range of equivalents to which such claims are entitled.

In the foregoing description, if various features are grouped together in a single embodiment for the purpose of streamlining the disclosure, this method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims, and such other claims as may later be added, are hereby incorporated into the description of the embodiments of the invention, with each claim standing on its own as a separate preferred embodiment.

We claim:

1. A method for producing oxygen, hydrogen and a hydrocarbon from carbon dioxide and hydron-containing extraterrestrial materials including the steps of:
exposing dodecatungstophosphoric acid to carbon dioxide and water in a reaction vessel;
allowing pH and pressure to drop in a series of one or more steps; and
removing hydrogen and oxygen from the vessel.

2. The method of claim 1, including a subsequent step of synthesizing methane from carbon dioxide and hydrogen.

3. The method of claim 1, wherein hydrogen is extracted from regolith.

4. The method of claim 1, wherein carbon dioxide is collected from an atmosphere of a celestial body.

5. The method of claim 1, including steps of:
blending polycarbonate with PO₄W₁₂O₃₆ to form a large complex polymer;
hydrogenating this polymer; and
introducing this polymer into CO₂ to form bicarbonates at tungsten sites.
